(19) United States
(12) Patent Application Publication
(10) Pub. No.: US 2010/0292681 A1
(43) Pub. Date: Nov. 18, 2010

(54) DEVICE FOR THE USE, ALSO SINGLE USE, OF AN OPTICAL FIBER FOR INVASIVE SURGICAL LASER TREATMENT IN THE HUMAN BODY

(75) Inventors: Damiano Fortuna, Rufina (IT); Leonardo Masotti, Sesto Fiorentino (IT); Andreas Papini, Livorno (IT)

Correspondence Address: MCGLEW & TUTTLE, PC, P.O. BOX 9227, SCARBOROUGH STATION, SCARBOROUGH, NY, 10510-9227, US

(73) Assignee: EL.EN. S.P.A., Calenzano, Firenze (IT)

(21) Appl. No.: 12/681,195

(22) PCT Filed: Oct. 3, 2008
(86) PCT No.: PCT/IT2008/000627

§ 371 Date: Jul. 1, 2010

(30) Foreign Application Priority Data
Oct. 5, 2007 (IT) FI2007A000221

(51) Int. Cl. A61B 18/22

(52) U.S. Cl. 606/15

(57) ABSTRACT

The device comprises a distal portion of rigid tube and a proximal portion of flexible tube connected to one another, in which the optical fiber is housed and fixed, the desired useful end of this fiber projecting from said portion of rigid tube; a manoeuvring tubular handpiece is fitted on said portion of rigid tube.

### TECHNICAL FIELD

The invention concerns a device for performing intentionally invasive treatments for lysis of adipocytes and for other minimally invasive surgical operations, such asskin tightening, laser interstitial thermal therapy, or laser ablation, percutaneous thermal treatment of confined neoplastic masses.

### BACKGROUND OF THE INVENTION

Laser energy is used in several medical applications. Some of said applications are percutaneous, i.e. the laser beam is delivered under the skin of the patient by means of a pervious needle, a cannula or the like.

U.S. Pat. Nos. 5,954,710 and 6,206,873 describe laser devices and methods for removal of adipose layers, i.e. for performing lipolysis by means of laser energy by conveying laser energy by means of an optical fiber guided through a cannula. The fiber is brought in the adipose tissues under the patient's skin and moved back and forth to cause lysis of the adipose cells. The device is used to treat also relatively extended areas with a beam of laser energy through an optical fiber.

Devices for percutaneous laser treatments are described in U.S. Pat. No. 6,574,401, and also in other documents mentioned therein. Other similar devices, but for ophtalmology, are disclosed in U.S. Pat. No. 5,603,710 and U.S. Pat. No. 6,015,403. Devices for intravascular treatments are disclosed in FR 2875122.

These known devices for laser treatment are particularly complex.

### SUMMARY OF THE INVENTION

The object of the invention is to provide a simpler and more economical device.

In some embodiments, the device according to the invention substantially comprises:

a distal portion of rigid tube;

a proximal portion of flexible tube coupled to said distal portion of rigid tube;

an optical fiber housed and fixed in said two tube portions, the desired useful end of the optical fiber projecting from the distal end of the portion of rigid tube;

a tubular manoeuvring handpiece, fitted on said portion of rigid tube, from which handpiece projects a desired distal part of the portion of rigid tube; and

at the proximal end of the portion of flexible tube a dispensing assembly for dispensing laser energy to the optical fiber.

According to an advantageous embodiment of the invention, the device comprises a locking member, which can be applied to one end of the handpiece, the locking member, when axially fitted in one of the ends of the rigid portion of tube, being suitable to lock the handpiece in a desired position on the portion of rigid tube.

The portion of rigid tube and the portion of flexible tube can be connected by means of a heat-shrinkable tube.

### BRIEF DESCRIPTION OF THE DRAWINGS

The invention is easier to understand by following the description and attached drawing, which shows a non-limiting practical embodiment of the invention. In the drawing:

FIG. 1 is an overall view of the device;

FIGS. 2 and 3 are a detailed cross section view of the handpiece and of a possible locking member thereof, and a view of the line III-III of FIG. 2, respectively; and

FIG. 4 shows possible shapes of the distal end part of the optical fiber.

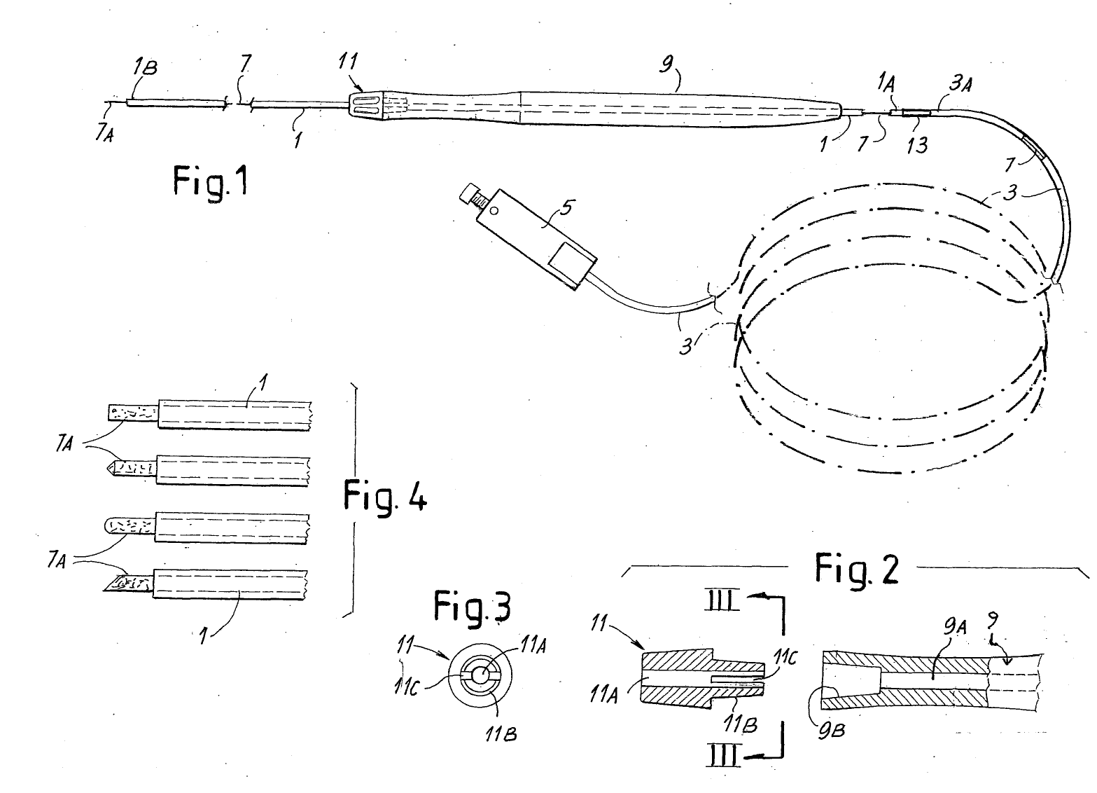

### DETAILED DESCRIPTION OF EMBODIMENTS OF THE INVENTION

According to what is illustrated in the drawing, number 1 indicates a relatively thin portion of a rigid tube or cannula, which is connected at the proximal end 1A with the distal end 3A of a portion of flexible tube 3, of considerable length. At its proximal end, said portion of flexible tube 3 is connected to a dispensing assembly for dispensing laser energy, generically indicated with the number 5, of a type known for surgical applications. The two portions of rigid tube 1 and of flexible tube 3 house an optical fiber 7, which can be fixed to one and/or to the other of said portion of tube 1 and 3, in such a way that the distal end 7A of said optical fiber 7 projects by a desired limited length from the distal end 1B of the tube 1. The optical fiber 7 originates from the dispensing assembly 5 for dispensing laser energy of a type suitable for surgical operations in general and in particular e.g. for the lysis of adipose cells, with a method as disclosed in U.S. Pat. No. 6,206,873.

In order to offer an easy grip for maneuvering the cannula formed by the tube portions 1 and 3 and in particular for maneuvering the portion of rigid tube 1, a handpiece 9 is provided, at least partially engaged to the portion of rigid tube 1. This handpiece will have an ergonomic shape for the various surgical operations.

A locking member 11 (see in particular FIGS. 2 and 3) can be adopted, which has a through hole 11A developed also in an elongation 11B slightly tapered and provided with at least two slits 11C, said elongation 11 being suitable to be force-fitted in a corresponding end housing 9B of the handpiece 9. Force fitting in this housing also causes the at least two parts, into which said elongation 11N is subdivided by the slits 11C, to move slightly towards each other.

In the holes 9A and 11A, or at least in the hole 11A is housed the portion of rigid tube 1 which in turn contains the optical fiber 7. Insertion and force-fitting of the elongation 11B of the locking member in the housing 9B causes a contraction of the slits 11C and therefore clamping of the locking member 11 and of the handpiece 9 with respect to the portion of rigid tube 1 in the desired position of the handpiece 9 on this portion of rigid tube 1; the length of said portion of rigid tube 1 which projects from the handpiece 9 can be easily determined.

The end 7A of the optical fiber 7 can be shaped in one of the ways illustrated by way of example in FIG. 4. The lateral surface of said end 7A of the optical fiber 7 can have suitable abrasions or other treatments, suitable to allow also lateral diffusion of laser light energy coming from the optical fiber itself.

The optical fiber 7 is fixed inside one and/or the other of the tubes 1 and 3 with any glue or with any other means, in any case so as to guarantee the desired projection of the end 7A of the optical fiber. The end 7A of the optical fiber 7 can be made to project by a length in the order of 0.5 mm to 10 mm.

The connection, indicated with the number 13, between the portion of rigid tube 1 and the portion of flexible tube 3 can be obtained by using a heat-shrinkable tube.

The portion of rigid tube 1 can be made of metal, for example stainless steel, or other biocompatible plastic material. The portion of flexible tube 3 can be made of plastic material such as so-called Teflon®, or silicone or other.

The various components can be made of materials suitable for single use, i.e. the device can be disposable, or entirely or in part of materials suitable to be used repeatedly after resterilization.

The lengths may be: the lengths of the portion of rigid tube 1 in the order of 10 to 40 mm; the length of the portion of the flexible tube 3 in the order of approximately 100 mm and over; or partly of the quantity suitable for the specific surgical operation. In this way, a single device can be adapted to the various types of operation, without the need for devices with cannulas of different length for the surgical treatment to be performed.

The rigid tube 1 can be made to project almost completely from the handpiece 9, in order to allow wide excursions inside the body mass to be treated. The fiber may have diameters ranging from 100 micron to 1 mm

It is understood that the drawing only shows an example provided by way of a practical arrangement of the invention, which can vary in forms and arrangement without however departing from the scope of the concept underlying the invention. Any reference numerals in the appended Claims are provided for the sole purpose of facilitating reading in the light of the description and the drawing, and do not in any manner limit the scope of protection represented by the claims.

1. A device for invasive surgical laser operations, the device comprising:
a tube in which an optical fiber is housed, wherein a distal end of said fiber projects from a distal end of said tube;
a maneuvering handpiece at least partially engaged on said tube, from which handpiece a part of the tube projects, wherein said tube comprises a distal rigid tube portion and a proximal flexible tube portion connected to each other, said optical fiber being fixed in said tube such that said optical fiber projects from the distal end of the rigid tube portion by a desired limited length, said handpiece being engaged on said rigid tube portion, said distal end of said rigid tube portion projecting from said handle.

2. A device as claimed in claim 1, further comprising a dispensing assembly for dispensing laser energy to the optical fiber, said dispensing assembly being arranged at the proximal end of the flexible tube portion.

3. A device as claimed in claim 1, wherein the device is disposable.

4. A device as claimed in claim 1 further comprising a locking member which can be applied to one end of the handpiece and which is suitable to lock the handpiece in a desired position on the rigid tube portion.

5. A device as claimed in claim 4, wherein said handpiece is tubular.

6. A device as claimed in claim 1, wherein said handpiece has an ergonomic shape for different surgical operations.

7. A device as claimed in claim 1, wherein said rigid tube portion and said flexible tube portion are connected by a heat-shrinkable tube.

8. A device as claimed in claim 1, wherein the lateral surface of the projecting distal end of said optical fiber presents abrasions or other treatments suitable to release laser energy.

9. A device as claimed in claim 2, wherein the device is disposable.

10. A device as claimed in claim 2, further comprising a locking member which can be applied to one end of the handpiece and which is suitable to lock the handpiece in a desired position on the rigid tube portion.

11. A device as claimed in claim 10, wherein said handpiece is tubular.

12. A device as claimed in claim 1, wherein said fiber is fixed in said rigid tube portion or said flexible tube portion by means of glue.
